(19) United States
(12) Patent Application Publication
(10) Pub. No.: US 2023/0226304 A1
(43) Pub. Date: Jul. 20, 2023

(54) NON-INVASIVE VENTILATION SYSTEM FOR THE PRE-HOSPITAL MANAGEMENT OF ACUTE RESPIRATORY FAILURE

(71) Applicants: OSPEDALE SAN RAFFAELE S.R.L., Milano (MI) (IT); POLITECNICO DI MILANO, Milano (MI) (IT)

(72) Inventors: Giovanni LANDONI, Milano (MI) (IT); Alberto ZANGRILLO, Milano (MI) (IT); Filippo CONSOLO, Milano (MI) (IT); Alberto REDAELLI, Milano (MI) (IT); Gianfranco Beniamino FIORE, Milano (MI) (IT); Alessandro CAIMI, Milano (MI) (IT)

(73) Assignees: OSPEDALE SAN RAFFAELE S.R.L., Milano (MI) (IT); POLITECNICO DI MILANO, Milano (MI) (IT)

(21) Appl. No.: 17/997,715

(22) PCT Filed: May 4, 2021
(86) PCT No.: PCT/EP2021/061722

§ 371 Date: Nov. 1, 2022

(30) Foreign Application Priority Data
May 4, 2020 (IT) 102020000009712

(51) Int. Cl. A61M 16/10; A61M 16/00; A61M 16/20

(52) U.S. Cl. CPC A61M 16/107; A61M 16/024; A61M 16/0066; A61M 16/205; A61M 16/209; A61M 2205/8206

(57) ABSTRACT

A stand-alone continuous positive airways pressure, CPAP, apparatus having a face-mask and a connected electro-mechanical device to supply air to the face-mask is disclosed. The electro-mechanical device includes a pneumatic channel for flowing air to be delivered to the face mask and a control unit for managing the air pressure of the air inside the pneumatic channel. The CPAP apparatus includes a turbine fan, located in the electro-mechanical device housing, connected to the control unit for pressurizing atmospheric air. The pneumatic channel includes an inlet portion located upstream of the turbine fan to receive atmospheric air, and an outlet portion located downstream of the turbine fan to deliver the pressurized air to the face-mask through an outlet opening. The pneumatic channel also longitudinally extends from the inlet portion to the outlet portion.

### TECHNICAL FIELD

The present invention relates to a medical system and particularly to a stand-alone continuous positive airways pressure, CPAP, apparatus. Also, the present invention relates to a non-invasive ventilation system.

### BACKGROUND

Respiratory failure is a severe, life-threatening medical emergency that affects every year >10% of the population and is characterized by high mortality (>20%). Its prevalence is growing incessantly; a recent survey by the Istituto Superiore di Sanità estimated that respiratory failure will become the third cause of death in Italy by 2020. It represents a severe burden to the healthcare system, with >100,000 hospitalizations/year, accompanied by prolonged hospital stay in the intensive care unit (ICU) for advanced and expensive therapies (>1 million hospital days/year). Considering that 75% of healthcare costs for respiratory failure are linked to hospitalization in the highly specialized ICU, anticipating the intervention and improving the prognosis will result in a reduced need for hospitalization and/or prolonged hospital stay, i.e., reduction of the costs associated with the treatment of the disease.

To contrast respiratory failure, techniques employing continuous positive airway pressure (CPAP) are used which apply mild air positive pressure on a continuous basis. It keeps the airways continuously open in people who are able to breathe spontaneously on their own, but need help keeping their airway unobstructed.

To date, CPAP is confined in the hospital ward, preferably in the ICUs. ICU-CPAP systems are not intended for out-of-hospital use, due to intrinsic technological features that prevent their use by the non-medical population.

Previous experiences in different EU countries demonstrated that pre-hospital CPAP is a life-saving procedure, even in the absence of active ventilation, consistent with improved chances of patient survival, and significant reduction of long-time complications.

The only commercially available external CPAP systems are limited to home treatment of obstructive sleep apnea. Therefore, these systems are intended for different purposes compared to the systems used in the ICUs. Furthermore, these are expensive and of difficult setting for a non-medical population. In fact, the setting requires the expertise of a pulmonologist.

It is therefore an object of the present invention to provide a system that solves the abovementioned problems, in particular, to provide a stand-alone CPAP apparatus that is easy-to-use, i.e. that can be operated by the non-medical population, that is not expensive, and with extended capabilities compared to commercial external CPAP systems.

This object is achieved by the stand-alone CPAP apparatus and the non-invasive ventilation system according to the independent claims. Further advantageous combinations and designs are given in the dependent claims therefrom.

### DESCRIPTION OF THE INVENTION

The stand-alone CPAP apparatus according to the present invention serves to contrast respiratory failure of a patient. The apparatus comprises a face-mask to deliver continuous pressured air to the patient's airways, and an electro-mechanical device having a housing directly and rigidly connectable to the face-mask to supply air to said face-mask at a controlled pressure value. The electro-mechanical device comprises a pneumatic channel for flowing air to be delivered to the face-mask and a control unit for automatically managing the value of the pressure of the air inside the pneumatic channel.

In particular, the apparatus comprises a turbine fan connected to the control unit and located in the housing of the electro-mechanical device for pressurizing atmospheric air to a determined pressure level value and the pneumatic channel includes an inlet portion located upstream of the turbine fan to receive atmospheric air, and an outlet portion located downstream of the turbine fan to deliver the pressurized air to the face-mask through an outlet opening. The pneumatic channel extends longitudinally in the housing from the inlet portion to the outlet portion. Advantageously, the turbine fan works in a pressure range between 5 and 10 cmH2O (490-980 Pa). For the purpose of the present invention, similar fans and/or any other type of fan that is configured to avoid unwanted overpressure in the apparatus can be used. For example, in an non-exhaustive list, the turbine fan can be a radial/centrifugal fan, a cross-flow fan, a tangential fan, auger fan or an axial fan.

With respect to prior art CPAP systems, the apparatus according to the present invention is more compact and easily maneuverable. In fact, the particular configuration according to which the housing of the electro-mechanical device is rigidly and directly connectable to the face mask makes the apparatus according to the present invention a stand-alone device that can be easily applied to the face of a person in any situation of emergency, for example in ambulance during the transportation of the patient to the hospital or for out-of-hospital use, such as in a public place (subways, supermarkets, schools, on the street) or in the private residence of the patient. Also, the apparatus according to the present invention can be used by any person without a specific medical knowledge. In fact, no additional/external console or power or control units are required. It is evident that such an invention can be extremely useful in order to avoid wasting precious time during a respiratory failure of a person, while waiting for intervention of paramedical or medical staff. In other words, the apparatus according to the present invention is conceived for the early pre-hospital management of acute respiratory failure via non-invasive ventilation in out-of-hospital environment, either at home or in public access areas. The apparatus applies oxygen-free continuous positive airway pressure to the airways of a patient. The present invention can be used similarly to the automated external defibrillators (AEDs), which provided evidence that, in the case of a medical emergency, pre-hospital intervention operated by the non-medical population is feasible and highly effective. In a basic configuration, the face-mask is applied to the airways of the patient (by the patient itself or by a different person both medical or non-medical) by simply holding the housing of the electro-mechanical device using one hand. The rigid configuration of the entire apparatus guarantees a correct position of the mask on the patient's face. However, to better fix the mask to the patient's airways, the mask can comprise connection elements such as for example an elastic strip to be applied around the head of the patient. In this way, it is not necessary holding the housing of the apparatus with the hand during the operation.

The activation of the turbine fan can occur manually or automatically. In the first case, the user can activate the functioning of the fan, thereby generating overpressure air inside the apparatus by using a dedicated actuator (switch) present on the external surface of the housing. Alternatively or in combination, the turbine fan can be automatically activated when the apparatus is removed from a dedicated case. For example, the apparatus can be located inside a glass case, where could be continuously supplied by an electric line, during a non operation condition. In an emergency situation, the user can simply take the apparatus from the case and directly apply the mask to the face of a person having breathing failure without worrying to switch the apparatus on. In fact, the turbine fan is automatically activated once the apparatus is removed from the case.

The apparatus can advantageously deliver pressurized air in a biphasic/bi-level positive air pressure (BiPAP) mode.

Additionally, the apparatus can be configured to deliver oxygen to the user.

Despite over-simplification of its layout, the apparatus according to the present invention reproduces the lifesaving “operating principle” of ICU-CPAP units, i.e., delivery of continuous positive airway pressure. In addition, the present invention profoundly innovates current therapeutic chain of acute respiratory failure. Indeed, the deployment of this apparatus over the territory will allow to anticipate the current therapeutic intervention of acute respiratory failure, enhancing prompt intervention by the non-medical population outside of the hospital setting. The aim of the apparatus is to improve the prognosis of a large number of fragile individuals, before the arrival of the emergency unit and/or patient's hospital admission. Importantly, it will facilitate more rapid clinical recovery and the return to normal life activities, reducing the risks and prevalence of long-term disability. In view of this, the apparatus according to the present invention will contribute to helping individuals suffering from respiratory failure to stay socially and economically active. Moreover, the present stand-alone CPAP apparatus will considerably reduce the financial impact of the disease and will enhance the sustainability of the healthcare system.

In one embodiment, the apparatus can comprise connection means located between the face-mask and the housing of the electro-mechanical device for allowing the face-mask to be detachable from said housing. The connection means preferably comprise a snap-fit fastening mechanism, a screwing mechanism, or any other suitable connection mechanism. It is noted that the possibility of detaching the face-mask from the housing of the electromechanical device can be extremely useful in terms of cost saving and reducing contamination. In fact, the face-mask is the only part of the apparatus in contact with the airways of the patient. The electro-mechanical device, on the other hand, is neither in contact with the face nor with other parts of the patient, for example the head. The only contact with the users would be in principle with their hand when they hold the apparatus. In this way, after replacing an already utilized face-mask with a new mask or a sterilized mask, the same electro-mechanical device can be used for a different patient, without the risk of contamination. In other words, the apparatus can be used for different patients in a very short time by simply substituting the face-mask. The only component that is replaced would be the face-mask that has a very reduced cost of production compared to the electro-mechanical device.

According to an embodiment, the turbine fan can be located outside the pneumatic channel that delivers pressurized air to the face-mask and then to the patient. Advantageously, not only the turbine fan but also the corresponding motor can be located outside the pneumatic channel. In this way, the risk of breathing toxic fumes that might potentially generate during the fan rotation and/or its motor operation (due for example to evaporation of the lubrication of the bearings and/or friction of internal parts) is minimized. Also, this configuration minimizes the risk of breathing heated air, since heating of the pressurized air might occur during the fan rotation and/or its motor operation.

As mentioned above, the pneumatic channel extends longitudinally between the inlet portion and the outlet portion. The pneumatic channel has therefore a central longitudinal axis passing through the outlet opening. In particular, the turbine fan can be located at a predetermined distance from said central longitudinal axis. The distance or the shift of the turbine fan from the central axis is determined to avoid a direct conduction of toxic fumes or heated air from the turbine fan to the outlet portion, as explained above.

In a specific example, the turbine fan can have a rotational axis parallel to the central longitudinal axis of the pneumatic channel, wherein the rotational axis is shifted from said central longitudinal axis. In other words, the turbine fan and the pneumatic channel are not coaxial. Alternatively, the rotational axis of the turbine fan can be oriented in a different direction compared to the central axis of the pneumatic channel. This could avoid the direct conduction of toxic fumes or heated air from the turbine fan to the outlet portion in a more effective way.

It is noted that alternatively the turbine fan can be located inside the pneumatic channel. In this way, the entire structure results more symmetric and compact. In an embodiment, the turbine fan can be located inside the pneumatic channel, whereas the rotational axis of the turbine fan can be oriented in a different direction compared to the central axis of the pneumatic channel.

In a further embodiment, the housing at the outlet portion can have an end region having a tapered shape. This can furthermore help in avoiding a direct conduction of toxic fumes or heated air from the turbine fan to the outlet portion. In fact, the pressurized air generated by the turbine fan would first hit the tapered walls of the end region of the housing before exiting from the outlet portion.

According to an embodiment of the present invention, the face-mask comprises a mechanical exhaust valve for automatically opening an air connection to the atmosphere in case the pressure level value in the pneumatic channel and then in the face-mask exceeds a pre-set threshold pressure value. The pre-set threshold value can be comprised between 15 and 20 cmH2O (1471-1961 Pa). It is noted that the face-mask is configured to ensure no or minimum air escape or leakage from the adhering surface to the patient's face. It is also noted that the present apparatus is able to provide different levels of CPAP and/or (positive end-expiratory pressure (PEEP), such as for example 5 cmH2O, 7 cmH2O, or 10 cmH2O.

In another embodiment of the present invention, the face-mask comprises at least one vent port to vent pressurized air during patient expiration. The vent port ensures that exhaled air is flushed away; as such, it allows the removal of carbon dioxide (CO₂) from the air-volume comprised in the mask, preventing CO₂ rebreathing.

In another embodiment of the present invention, the electro-mechanical device comprises a first filter located upstream of the turbine fan to protect the turbine fan from external contaminants. For example, the first filter can be a coarse filter to protect the fan from damages, e.g. caused by large air particulate, liquid drops or other macroscopic contaminants from the environment. In particular, the filter can be a EPA (Efficiency Particulate Air) filter.

In still another embodiment of the invention, the electro-mechanical device comprises a second filter located downstream of the turbine fan to purify the pressured air to be delivered the face-mask. In particular, the second filter can be a high efficiency particulate air, HEPA, filter or a similar filter.

In a further embodiment, the electro-mechanical device comprises a controlled exhaust valve for opening an air connection to the atmosphere, the controlled exhaust valve being located at the outlet portion of the pneumatic channel and being connected to the control unit. The controlled exhaust valve can act as a redundant safety element for overpressure prevention in addition to the mechanical exhaust valve.

In an other embodiment, the electro-mechanical device comprises a first pressure sensor and a second pressure sensor, independent from the first pressure sensor, both pressure sensors being located at the outlet portion of the pneumatic channel and being connected to the control unit to measure the pressure value in the pneumatic channel.

The control unit automatically manages the normal and abnormal pneumatic conditions of the apparatus. In the present case, normal and abnormal pneumatic conditions are intended as pressure values measured inside the pneumatic channel that are below or above a threshold value, for example the above-mentioned pre-set threshold value, respectively. In normal operating conditions, the control unit, by means of a fan speed control unit, is configured to adjust the pressure value inside the pneumatic channel by modifying the speed of the turbine fan based on the pressure value measured by a pressure sensor, e.g. the first pressure sensor. If abnormal overpressure takes place, the control unit, by means of an overpressure detector, is configured to activate the controlled exhaust valve and to stop the action of the turbine valve based on the pressure value measured by a different pressure sensor, e.g. the second pressure sensor.

According to an embodiment of the present invention, the electro-mechanical device further comprises a rechargeable battery pack to supply at least the control unit and the turbine fan. In particular, the control unit can be supplied by a low-voltage supply line (i.e. 24 V-DC) backed-up by a small battery pack for short-time stand-alone needs. Alternatively or additionally, the apparatus can be configured to be directly supplied by the power grid.

Advantageously, the face-mask can be made of a scented material or can be provided with scent diffusing means to improve the comfort of the patient during the breathing assisted operation.

The non-invasive ventilation system according to the present invention comprises the apparatus according to any of the preceding embodiments and a diagnostic and power supply unit connected to said apparatus. The diagnostic and power supply unit is configured for supplying current to a battery unit of the apparatus, for example the rechargeable battery pack, and for displaying information on the actual status of the apparatus components. In this way, it is possible to continuously monitor the conditions of the components of the apparatus, such as the battery charge status or a malfunctioning of the turbine fan.

Preferred embodiments of a stand-alone CPAP apparatus and the non-invasive ventilation system in accordance with the invention will be explained herein below in greater detail with reference to the accompanying drawings, in which:

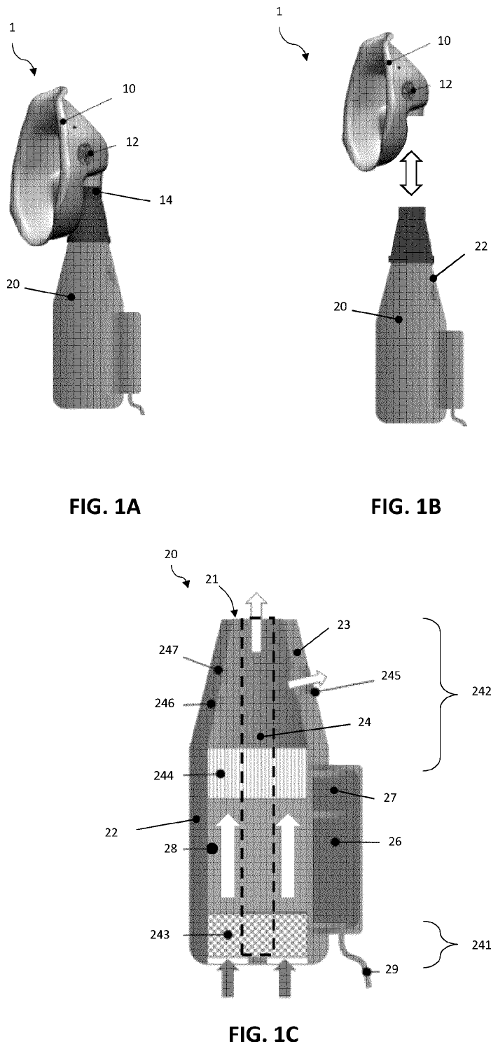

FIGS. 1a, 1b and 1c show a perspective view of the stand-alone CPAP apparatus according to the present invention in an assembled configuration with a face-mask (a), in a disassembled configuration (b), and in a section configuration without the face-mask (c);

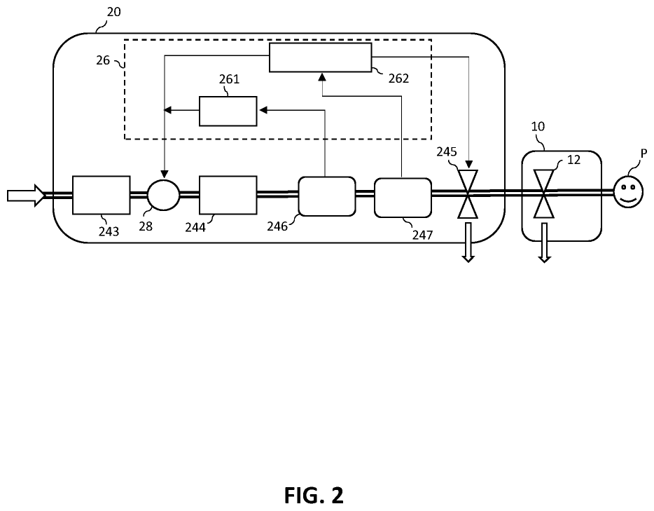

FIG. 2 shows a block diagram of the apparatus;

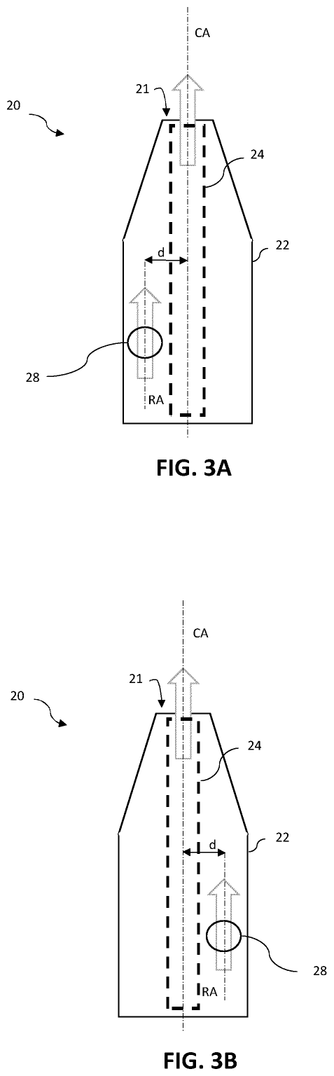

FIGS. 3a and 3b show a schematic representation of the position of the turbine fan with respect to the pneumatic channel in the housing of the apparatus; and

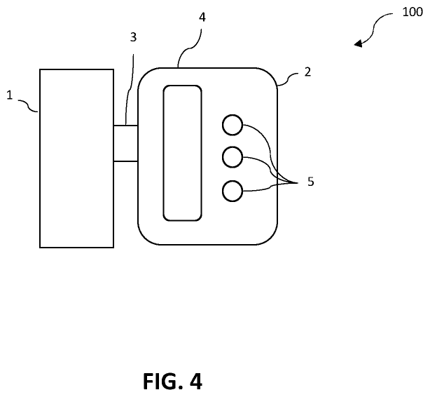

FIG. 4 shows a non-invasive ventilation system according to the present invention.

FIG. 1a describes a schematic representation of a stand-alone CPAP apparatus 1 according to the present application. The apparatus 1 comprises a face-mask 10 and an electro-mechanical device 20 connected to the face-mask 10. It is noted that the electro-mechanical device 20 is directly connected to the face-mask 10 in a rigid fashion. In other words, between the mask 10 and the electro-mechanical device 20 are not interposed any kind of flexible elements, such as electrical cable, or flexible tubes or ducts. In this way, the apparatus 1 is a stand-alone apparatus that can easily be handle also with a single hand. FIG. 1a also illustrates the presence of a mechanical exhaust valve 12 located on a side of the face-mask 10 acting as a safety means in case of overpressure.

The face-mask 10 can easily be detached by the electro-mechanical device 20, or rather by the housing 22 of said device 20, as illustrated in FIG. 1b. For this purpose, connection means 14 are located between the face-mask 10 and the device 20. The face-mask 10 can be detached and reattached to the housing 22 of the electro-mechanical device 20 thanks to, for example, a snap-fit fastening mechanism. Sealing elements are provided at the connection means for avoiding air escape from this conjunction region.

FIG. 1c describes the longitudinal section of the electro-mechanical device 20 of FIGS. 1a and 1b. The electro-mechanical device 20 includes a housing 22 that is directly connected to the face-mask 10. In particular, the electro-mechanical device 20 comprises a pneumatic channel 24 configured to allow the flowing of the air from the external environment to the face-mask 10, the pneumatic channel 24 having an inlet portion 241 for receiving atmospheric air from outside (two dark arrows on the bottom in FIG. 1c) and an outlet portion 242 for delivering pressurized air to the face-mask 10 through the outlet opening 21 (single white arrow on the top in FIG. 1c). It is noted that the housing 22 has the shape of a bottle, wherein at the outlet portion 242 the housing 20 has an end region 23 having a tapered shape. In the housing 22 is located a turbine fan 28 for pressurizing the atmospheric air received from outside at a predetermined pressure value. Upstream of the turbine fan 28 is located a coarse filter 243 to avoid the damage of the fan 28 caused by macroscopic contaminants. Downstream of the turbine fan 28 is on the other hand located a HEPA filter 244 to purify the pressurized air to be delivered to the face-mask 10.

The electro-mechanical device 20 further comprises a first pressure sensor 246 and a second pressure sensor 247. Both sensors are used to measure the pressure value inside the pneumatic channel 24 and are located downstream of the turbine fan 28 at the outlet portion 242 of the pneumatic channel. In addition, the electro-mechanical device 20 comprises a controlled exhaust valve 245 for opening an air connection to the atmosphere (with side arrow in FIG. 1c). The controlled exhaust valve 245 is located at the outlet portion 242 of the pneumatic channel 24.

At a side of the pneumatic channel 24, the electro-mechanical device 20 further comprises a control unit 26. The control unit 26 automatically manages the normal and abnormal pneumatic conditions of the apparatus. For this reason, the two pressure sensors 246, 247, the controlled exhaust valve 245, as well as the turbine fan 28 are connected to the control unit 26.

As shown in FIG. 1c, the control unit 26 is supplied by a low-voltage supply line 29, backed-up by a battery pack 27 for short-time stand-alone needs.

FIG. 2 illustrates a functional schematic representation of the electro-mechanical device 20 connected to the face-mask 10. Atmospheric air (horizontal arrow on the left side of FIG. 2) is introduced into the apparatus 1 and is filtered by the coarse filter 243. After passing through the turbine fan 28, the pressurized air is additionally filtered by the HEPA filter 244.

In normal operating mode, the control unit 26 is configured to adjust the pressure value inside the pneumatic channel 24 by modifying the speed of the turbine fan 28 based on the pressure value measured by the first pressure sensor 246. For this purpose, the control unit 26 comprises a fan speed control unit 261 connected to the turbine fan 28 and the first pressure sensor 246. Based on the information (i.e. the measured pressure value) received by the first pressure sensor 246, the fan speed control unit 261 acts on the speed of the turbine fan 28, for example by decelerating its functioning until reaching a controlled pressure value inside the pneumatic channel 24. At this point, the pressured air is first delivered to the face-mask 10 and then to the patient P.

In abnormal overpressure operating mode, the control unit 26 is configured to activate the controlled exhaust valve 245 and to stop the action of the turbine valve 28 based on the pressure value measured by the second pressure sensor 247. For this purpose, the control unit 26 comprises an overpressure detector 262 connected to the turbine fan 28, the controlled exhaust valve 245 and the second pressure sensor 247. Based on the information (i.e. the measured pressure value) received by the second pressure sensor 247, the overpressure detector 262 activates the controlled exhaust valve 245 and acts on the speed of the turbine fan 28, for example stopping its functioning. In this case, the over-pressurized air is expelled by the controlled exhaust valve 245 (vertical arrow at the controlled exhaust valve 245).

As an additional safety control element, before the pressurized air is delivered to the patient P, the face-mask 10 is provided with a mechanical exhaust valve 12. In case the pressure value of the air inside the mask is above a pre-set threshold value, the over-pressurized air is expelled by the mechanical exhaust valve 12 (vertical arrow at the controlled exhaust valve 12).

FIGS. 3a and 3b show a schematic representation of the housing 22 of the electro-mechanical device 20 and in particular the relative position of the turbine fan 28 with respect to the pneumatic channel 24. From the figures it is clear that the pneumatic channel 24 can be represented by a duct connecting the inlet portion to the outlet portion of the housing 22. In this particular case, the channel 24 is a longitudinal channel extending basically in the central internal part of the housing 22. According to the two configurations illustrated in the figures, the turbine fan 28, schematically depicted as a circle, is located outside the pneumatic channel 24. FIGS. 3a and 3b shows two possibilities, wherein the turbine fan 28 is located on the left side or the right side of the pneumatic channel 24. However, it is clear that the turbine fan 28 can be positioned in any region inside the housing 22 that is outside the channel 24. In particular, the pneumatic channel 24 has a central longitudinal axis CA that passes through the outlet opening 21 of the housing 22. Accordingly, the turbine fan 28 is located at a predetermined distance d from the central longitudinal axis CA. The value of the distance is determined to avoid a direct conduction of toxic fumes or heated air from the turbine fan 28 to the outlet opening 21.

It is noted that the rotational axis RA of the turbine fan 28 is parallel to the central longitudinal axis CA of the pneumatic channel 24 and the rotational axis RA is shifted from the central longitudinal axis CA. In particular, the distance d between the two parallel axes is determined to avoid a direct conduction of toxic fumes or heated air from the turbine fan 28 to the outlet opening 21. Although not shown in the figure, the rotational axis RA of the turbine fan 28 can be oriented in a different direction compared to the central axis CA of the pneumatic channel 24. This configuration can avoid the direct conduction of toxic fumes or heated air from the turbine fan 28 to the outlet opening 21 as well.

FIG. 4 illustrates a schematic representation of a non-invasive ventilation system 100 for the pre-hospital management of acute respiratory failure. This system can be located in a public place outside an hospital structure, such as a subways, a school, university, etc. The system 100 comprises the apparatus 1 as described in FIGS. 1a, 1b and 1c and a diagnostic and power supply unit 2 that is connected to the apparatus through connection means 3, for example electrical cables. The diagnostic and power supply unit 2 is configured to supply current to a battery unit of the apparatus 1. Also, this unit 2 is configured for displaying information on the actual status of the apparatus components. To this purpose, the diagnostic and power supply unit 2 comprises a display 4 and control lights 5 for informing the user on the charge status of the battery 27 inside the apparatus 1 or on possible malfunctioning of some components of the apparatus 1, such as the control unit 26, the turbine fan 28, the battery 27, the sensors 246, 247, the valve 245, etc.

Whilst features have been presented in combination of the above description, this is intended solely as an advantageous combination. The above description is not intended to show required combinations of features, rather it represents each of the aspects of the disclosure. Accordingly, it is not intended that any described specific combination of features is necessary for the functioning of the stand-alone CPAP apparatus or the non-invasive ventilation system.

1. A stand-alone continuous positive airways pressure, CPAP, apparatus comprising:
a face-mask to deliver continuous pressured air to a patient's airways, and
an electro-mechanical device having a housing directly and rigidly connectable to the face-mask to supply air to said face-mask at a controlled pressure value, the electro-mechanical device comprising a pneumatic channel for flowing air to be delivered to the face-mask and a control unit for automatically managing the value of the pressure of the air inside the pneumatic channel,
wherein the apparatus comprises a turbine fan connected to the control unit and located in the housing of the electro-mechanical device for pressurizing atmospheric air to a determined pressure level value, and wherein the pneumatic channel includes an inlet portion located upstream of the turbine fan to receive atmospheric air and an outlet portion located downstream of the turbine fan to deliver the pressurized air to the face-mask through an outlet opening, the pneumatic channel extending longitudinally in the housing from said inlet portion to said outlet portion.

2. The apparatus of claim 1, further comprising connection means located between the face-mask and the housing of the electro-mechanical device for allowing the face-mask to be detachable from said housing, wherein the connection means optionally comprise a snap-fit fastening mechanism or a screwing mechanism.

3. The apparatus according to claim 1, wherein the turbine fan is located outside the pneumatic channel that delivers pressurized air to the face-mask and then to the patient.

4. The apparatus according to claim 1, wherein the pneumatic channel has a central longitudinal axis (CA) passing through the outlet opening and the turbine fan is located at a predetermined distance from said central longitudinal axis (CA).

5. The apparatus according to claim 4, wherein the turbine fan has a rotational axis (RA) parallel to the central longitudinal axis (CA) of the pneumatic channel and wherein said rotational axis (RA) is shifted from said central longitudinal axis (CA).

6. The apparatus according to claim 4, wherein the housing at the outlet portion has an end region having a tapered shape.

7. The apparatus according to claim 1, wherein the face-mask comprises a mechanical exhaust valve for automatically opening an air connection to the atmosphere in case the pressure level value in the pneumatic channel exceeds a preset threshold pressure value.

8. The apparatus of claim 1, wherein the electro-mechanical device comprises a first filter located upstream of the turbine fan to protect the turbine fan from external contaminants.

9. The apparatus of claim 1, wherein the electro-mechanical device comprises a second filter located downstream of the turbine fan to purify the pressured air to be delivered the face-mask wherein the second filter optionally is a high efficiency particulate air, HEPA, filter.

10. The apparatus of claim 1, wherein the electro-mechanical device comprises a controlled exhaust valve for opening an air connection to the atmosphere, the controlled exhaust valve being located at the outlet portion of the pneumatic channel and being connected to the control unit.

11. The apparatus of claim 1, wherein the electro-mechanical device comprises a first pressure sensor and a second pressure sensor, independent from the first pressure sensor, both pressure sensors being located at the outlet portion of the pneumatic channel and being connected to the control unit to measure the pressure value in the pneumatic channel.

12. The apparatus of claim 11, wherein the control unit comprises a fan speed control unit to adjust the pressure value inside the pneumatic channel by modifying the speed of the turbine fan based on the pressure value measured by the first pressure sensor.

13. The apparatus of claim 10, wherein the control unit comprises an overpressure detector to activate the controlled exhaust valve and to stop the action of the turbine fan based on the pressure value measured by the second pressure sensor.

14. The apparatus of claim 1, wherein the electro-mechanical device further comprises a rechargeable battery pack to supply at least the control unit and the turbine fan.

15. A non-invasive ventilation system comprising an apparatus of claim 1 and a diagnostic and power supply unit connected to the apparatus for supplying current to a battery unit of the apparatus and for displaying information on the actual status of the apparatus components.
